SEQUENCE LISTING (1 sequence)

SEQ ID NO: 1 - protein, 480 aa, Homo sapiens [sequence omitted]

(19) United States
(12) Patent Application Publication
(10) Pub. No.: US 2005/0014682 A1
(43) Pub. Date: Jan. 20, 2005

(54) Cell-free assay for insulin signaling

(76) Inventors: Mike Mueckler, St. Louis, MO (US); Richard Hresko, Chesterfield, MO (US); Haruhiko Murata, Rockville, MD (US)

Correspondence Address: THOMPSON COBURN, LLP, ONE US BANK PLAZA, SUITE 3500, ST LOUIS, MO, 63101, US

(21) Appl. No.: 10/621,485

(22) Filed: Jul. 16, 2003

(51) Int. Cl. A61K 38/48

(52) U.S. Cl. 514/7; 424/94.5

(57) ABSTRACT

A cell-free assay system, which reconstitutes components of the phosphatidyl-inositol 3-kinase-mediated insulin signaling pathway including phosphatidylinositol phosphate dependent kinase-2 (“PDK2”). Alternatively, a in vitro method for phosphorylating a protein kinase B on Serine 473 or Serine 474. The invention relates generally to an in vitro method of phosphorylating a protein kinase B (“PKB” or “Akt”), to an in vitro method of assessing insulin action, and to an in vitro method of identifying an agent or process that modulates insulin signaling or any cellular activity regulated or influenced by PKB, including cell growth, mitosis, apoptosis, fuel metabolism, and oncogenic transformation. Such an agent or process may be useful in treating insulin resistance, diabetes, obesity, cancer, and a number of other diseases.

### GOVERNMENTAL SUPPORT

This work was supported by the U.S. Department of Health and Human Services/National Institutes of Health R01 grant number DK38495. The U.S. Government has certain rights in this invention.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

1. Field of the Invention

The invention relates generally to an in vitro method of phosphorylating a protein kinase B (“PKB” or “Akt”), to an in vitro method of assessing insulin action, and to an in vitro method of identifying an agent or process that modulates insulin signaling or any cellular activity regulated or influenced by PKB, including cell growth, mitosis, apoptosis, fuel metabolism, and oncogenic transformation. Such an agent or process may be useful in treating insulin resistance, diabetes, obesity, cancer, and a number of other diseases.

2. Description of the Related Art

Insulin initiates multiple signaling pathways leading to numerous responses that regulate carbohydrate, fat, and protein metabolism (Saltiel, 2001). Hormone binding induces a conformational change in the insulin receptor that activates its intrinsic tyrosine kinase through an autophosphorylation mechanism. The activated receptor can then phosphorylate several intracellular protein substrates, most notably the insulin receptor substrate (“IRS”) proteins (White, 1998; White, 1994). Tyrosine-phosphorylated IRS proteins can recruit and activate the downstream effector PI-3 kinase, which generates phosphatidylinositol (3,4,5) trisphosphate (PIP3) using inositol-containing phospholipids resident in the plasma membrane as substrates (Shepherd, 1998). Many of the metabolic effects of insulin are absolutely dependent on PI-3 kinase activation. For example, insulin stimulation of glucose uptake via translocation of the glucose transporter isoform Glut4 is completely blocked by the PI-3 kinase inhibitor wortmannin (Clark, 1994).

The serine/threonine kinase called protein kinase B (“Akt” or “PKB”) has emerged as a critical mediator operating downstream of PI-3 kinase (Lawlor, 2001). The activity of Akt is stimulated by phosphorylation on two of its amino acid residues: (1) threonine 308 in the activation loop of the kinase catalytic domain; and (2) serine 473 in the hydrophobic carboxy-terminal domain (SEQ ID NO:1 depicts the sequence of a human Akt protein, which is provided to orient the skilled artisan to the relevant threonine and serine residues; Vanhaesebroeck, 2000). The phosphorylation of both residues is wortmannin-sensitive in vivo (Alessi, 1996). The protein kinase responsible for phosphorylating Akt on Thr308 is the recently identified phosphoinositide-dependent kinase 1 (PDK1) (Alessi, 1997a; Alessi, 1997b; Stephens, 1998). Despite intense investigative efforts, the kinase responsible for phosphorylating Akt on Ser473—tentatively termed phosphoinositide-dependent kinase 2 (PDK2)—has yet to be identified (Brazil, 2001; Toker, 2000a; Vanhaesebroeck, 2000). PDK1 (Balendran, 1999) and even Akt itself (by an autophosphorylation mechanism) (Toker, 2000b) have been proposed as possible candidates for PDK2. The search for the elusive PDK2 remains a major unresolved issue with regard to the regulation of Akt.

In vitro assays have proven to be enormously useful for many areas of biology, including the investigation of insulin action. During the late 1970's, L. Jarett and colleagues noted that the direct addition of insulin to a purified adipocyte plasma membrane fraction resulted in numerous effects, including alterations in the phosphorylation of several proteins (Seals, 1979) and increased calcium binding by the plasma membrane (McDonald, 1976). These investigators had very few guideposts available at the time for interpreting their observations in a molecular context; indeed, their work predated the cloning of the cDNA encoding the insulin receptor, which occurred in 1985 (Ebina, 1985; Ullrich, 1985). More recently, the laboratory of C. R. Kahn employed subcellular fractions of 3T3-L1 adipocytes to reconstitute (1) the dynamic association of IRS-1/2 and PI-3 kinase with various cellular compartments (Inoue, 1998), and (2) the binding of Glut4 vesicles to the plasma membrane (Inoue, 1999). These investigators employed components derived from cells that were treated in vivo with or without insulin (100 nM for 10 minutes at 37° C.). The extent of manipulations that can be performed in their assay may thus be potentially limited due to the likelihood of the insulin-dependent process under investigation having already occurred in vivo, prior to the time the components of their assay are recombined in vitro. This limitation can explain why the insulin-stimulated association of Glut4 vesicles with the plasma membrane that they observe in vitro is wortmannin-insensitive and does not require ATP or cytosol (Inoue, 1999).

3. Bibliography

The following bibliography pertains to references cited in all sections of this document. The inventors make no claim regarding the accuracy and pertinence of these references as prior art and reserve the right to challenge the accuracy of these references. All references cited herein are hereby incorporated by reference in their entirety.
Alessi, D. R., Andjelkovic, M., Caudwell, B., Cron, P., Morrice, N., Cohen, P., and Hemmings, B. A. (1996). Mechanism of activation of protein kinase B by insulin and IGF-1. EMBO J 15, 6541-6551.
Alessi, D. R., Deak, M., Casamayor, A., Caudwell, F. B., Morrice, N., Norman, D. G., Gaffney, P., et al. (1997a). 3-Phosphoinositide-dependent protein kinase-1 (PDK1): structural and functional homology with the Drosophila DSTPK61 kinase. Curr Biol 7, 776-789.
Alessi, D. R., James, S. R., Downes, C. P., Holmes, A. B.; Gaffney, P. R., Reese, C. B., and Cohen, P. (1997b). Characterization of a 3-phosphoinositide-dependent protein kinase which phosphorylates and activates protein kinase B. Curr Biol 7, 261-269.
Balendran, A., Casamayor, A., Deak, M., Paterson, A., Gaffney, P., Currie, R., Downes, C. P., and Alessi, D. R. (1999). PDK1 acquires PDK2 activity in the presence of a synthetic peptide derived from the carboxyl terminus of PRK2. Curr Biol 9, 393-404.
Behn-Krappa, A., and Newton, A. C. (1999). The hydrophobic phosphorylation motif of conventional protein kinase C is regulated by autophosphorylation. Curr Biol 9, 728-737.
Brazil, D. P. a. H., B. A. (2001). Ten years of protein kinase B signalling: a hard AKT to follow. TRENDS Biochem Sci 26, 657-664.
Calderhead, D. M., Kitagawa, K., Tanner, L. I., Holman, G. D., and Lienhard, G. E. (1990). Insulin regulation of the two glucose transporters in 3T3-L1 adipocytes. J Biol Chem 265, 13801-13808.
Clark, J. F., Young, P. W., Yonezawa, K., Kasuga, M., and Holman, G. D. (1994). Inhibition of the translocation of GLUT1 and GLUT4 in 3T3-L1 cells by the phosphatidylinositol 3-kinase inhibitor, wortmannin. Biochem J 300, 631-635.
Clark, S. F., Martin, S., Carozzi, A. J., Hill, M. M., and James, D. E. (1998). Intracellular localization of phosphatidylinositide 3-kinase and insulin receptor substrate-1 in adipocytes: potential involvement of a membrane skeleton. J Cell Biol 140, 1211-1225.
Coffer, P. J., Jin, J., and Woodgett, J. R. (1998). Protein kinase B (c-Akt): a multifunctional mediator of phosphatidylinositiol 3-kinase activation. Biochem. J. 335, 1-13.
Cross, D. A., Alessi, D. R., Cohen, P., Andjelkovich, M., and Hemmings, B. A. (1995). Inhibition of glycogen synthase kinase-3 by insulin mediated by protein kinase B. Nature 378, 785-789.
Currie, R. A., Walker, K. S., Gray, A., Deak, M., Casamayor, A., Downes, C. P., Cohen, P., Alessi, D. R., and Lucocq, J. (1999). Role of phosphatidylinositol 3,4,5 trisphosphate in regulating the activity and localization of 3-phosphoinositide—dependent protein kinase-1. Biochem J 337, 575-583.
Critchley, D. R. (2000) Focal adhesions—the cytoskeletal connection. Curr. Opin. Cell Biol. 12, 133-139.
Dehar, S., Williams, B. and Hannigan, G. (1999) Integrin-linked kinase (ILK): a regulator of integrin and growth-factor signalling. Trends Cell Biol. 9, 319-323.
Delcommenne, M., Tan, C., Gray, V., Ruel, L., Woodgett, J., and Dedhar, S. (1998). Phosphoinositide-3-OH kinase-dependent regulation of glycogen synthase kinase 3 and protein kinase B/AKT by the integrin-linked kinase. Proc Natl Acad Sci USA 95, 11211-11216.
Denu, J. M., and Tanner, K. G. (1998). Specific and reversible inactivation of protein tyrosin phosphatases by hydrogen peroxide: evidence for a sulfenic acid intermediate and implications for redox regulation. Biochemistry 37, 5633-5642.
Ebina, Y. E., Ellis, L., Jamagin, K., Edery, M., Graf, L., Clauser, E., Ou, J. H., Masiarz, F., Kan, Y. W., Goldfine, I. D., et al., (1985). The human insulin receptor cDNA: the structural basis for hormone-activated transmembrane signaling. CELL 40, 747-758.
Endemann, G., Yonezawa, K., and Roth, R. A. (1990). Phosphatidylinositol kinase or an associated protein is a substrate for the insulin receptor tyrosine kinase. J Biol Chem 265, 396-400.
Garen, A., and Levinthal, C. (1960). A fine-structure genetic and chemical study of the enzyme alkaline phosphatase of E. coli I. Purification and characterization of alkaline phosphatase. Biochim Biophys Acta 38, 470-483.
Gordon, J. (1991). Use of vanadate a as protein-phosphotyrosine phosphatase inhibitor. Methods Enzymol 201, 477-482.
Heller-Harrison, R. A., Morin, M., and Czech, M. P. (1995) Insulin regulation of membrane-associated insulin receptor substrate 1. J. Biol. Chem. 270, 24442-24450.
Hill, M. M., Clark, S. F., Tucker, D. F., Bimbaum, M. J., James, D. E., and Macaulay, S. L. (1999). A role for protein kinase B/AKT2 in insulin-stimulated GLUT4 translocation in adipocytes. Mol Cell Biol 19, 7771-7781.
Holgado-Madruga, M., Emlet, D. R., Moscatello, D. K., Godwin, A. K., and Wong, A. J. (1996). A Grb2-associated docking protein in EGF-and insulin-receptor signalling. Nature 379, 560-563.
Inoue, G., Cheatham, B., and Kahn, C. R. (1999). Development of an in vitro reconstitution assay for glucose transporter 4 translocation. Proc Natl Acad Sci USA 96, 14919-14924.
Inoue, G., Cheatham, B., Emkey, R., and Kahn, C. R. (1998). Dynamics of insulin signaling in 3T3L1 adipocytes. Differential compartmentalization and trafficking of insulin receptor substrate (IRS-1) and IRS-2. J Biol Chem 273, 11548-11555.
James, D. E., Strube, M., and Mueckler, M. (1989). Molecular cloning and characterization of an insulin-regulatable glucose transporter. Nature 338, 83-87.
Jarett, L. (1974). Subcellular fractionation of adipocytes. Methods Enzymol 31, 60-71.
Kelly, K. L., and Ruderman, N. B. (1993). Insulin-stimulated phosphatidylinositol 3-kinase. Association with a 185-kDa tyrosine phosphorylated protein (IRS-1) and localization in a low density membrane vesicle. J Biol Chem 268, 4391-4398.
Khwaja, A., Rodriguez-Viciana, P., Wennstrom, S., Wame, P. H., and Downward, J. (1997) Matrix adhesion and Ras transformation both activate a phosphoinositide 3-OH kinase and protein kinase B/Akt cellular survival pathway. EMBO 16, 2783-2793.
Kriauciunas, K. M., Myers, M. G., and Kahn, C. R. (2000) Cellular compartmentalization in insulin action: altered signaling by a lipid-modified IRS-1. Mol. Cell. Biol. 20, 6849-6859.
Lavan, B. E., and Lienhard, G. E. (1993). The insulin-elicited 60-kDa phosphotyrosin protein in rat adipocytes is associated with phosphatidylinositol 3-kinase. J Biol Chem 268, 5921-5928.
Lawlor, M. A., and Alessi, D. R. (2001). PKB/AKT: A key mediator of cell proliferation, survival, and insulin responses? J Cell Sci 114, 2903-2910.
Lynch, D. K., Ellis, C. A., Edwards, P. A. W., and Hiles, I. D. (1999). Integrin-linked kinase regulates phosphorylation of serine 473 of protein kinase B by an indirect mechanism. Oncogene 18, 8024-8032.
McDonald, J. M., Bruns, D. E., and Jarett, L. (1976). Ability of insulin to increase calcium binding by adipocyte plasma membranes. Proc Natl Acad Sci USA 73, 1542-1546.
Persad, S. A., S., Gray, V., Mawji, N., Deng, J. T., Leung, D., Yan, J. et al., (2001). Regulation of protein kinase B/AKT-serine 473 phosphorylation by integrin-linked kinase: critical roles for kinae activity and amino acids arginine 211 and serine 343. J Biol Chem 276, 27462-27469.
Piper, R. C., Hess, L. J., and James, D. E. (1991). Differential sorting of two glucose transporters expressed in insulin-sensitive cells. Am J Physiol 260, C570-C580.
Reed, B. C., Kaufman, S. H., Mackall, J. C., Student, A. K., and Lane, M. D. (1977). Alterations in insulin binding accompanying differentiation of 3T3-L1 preadipocytes. Proc Natl Acad Sci USA 74, 4876-4880.
Rice, K. M., Lienhard, G. E., and Garner, C. W. (1992). Regulation of the expression of pp160, a putative insulin receptor signal protein, by insulin, dexamethason , and 1-methyl-3-isobutylxanthine in 3T3-L1 adipocytes. J Biol Chem 267, 10163-10167.
Rubin, C. S., Lai, E., and Rosen, O. M. (1977). Acquisition of increased hormone sensitivity during in vitro adipocyte development. J Biol Chem 252, 3554-3557.
Ruderman, N. B., Kapeller, R., White, M. F., and Cantley, L. C. (1990). Activation of phosphatidylinositol 3-kinase by insulin. Proc Natl Acad Sci USA 87, 1411-1415.
Saltiel, A. R., and Kahn, C. R. (2001). Insulin signalling and the regulation of glucose and lipid metabolism. Nature 414, 799-806.
Schlemmer, S. R., and Sirotnak, F. M. (1992). Energy-dependent efflux of methotrexate in L1210 leukemia cells. Evidence for the role of an ATPase obtained with inside-out plasma membrane vesicles. J Biol Chem 267, 14746-14752.
Seals, J. R., McDonald, J. M. and Jarett, L. (1979). Insulin effect on protein phosphorylation of plasma membranes and mitochondria in a subcellular system from rat adipocytes. I. Identification of insulin-sensitive phosphoproteins. J Biol Chem 254, 6991-6996.
Shepherd, P. R., Withers, D. J., and Siddle, K. (1998). Phosphoinositide 3-kinase: th key switch mechanism in insulin signalling. Biochem J 333, 471-490.
Simpson, I. A., Yver, D. R., Hissin, P. J., Wardzala, L. J., Karnielli, E., Salans, L. B., and Cushman, S. W. (1983). Insulin-stimulated translocation of glucose transporters in the isolated rat adipose cells: characterization of subcellular fractions. Biochim Biophys Acta 763, 393-407.
Stephens, L., Anderson, K., Stokoe, D., Erdjument-Bromage, H., Painter, G. F., Holmes, A. B., Gaffney, P. R., et al. (1998). Protein kinase B kinases that mediat phosphatidylinositol 3,4,5-trisphosphate-dependent activation of protein kinase B. Science 279, 710-714.
Summers, S. A., Whiteman, E. L., Cho, H., Lipfert, L., and Bimbaum, M. J. (1999). Differentiation-dependent suppression of platelet-derived growth factor signaling in cultured adipocytes. J Biol Chem 274, 23858-23867.
Takakura, K., Beckman, J. S., MacMillan-Crow, L. A., and Crow, J. P. (1999). Rapid and irreversible inactivation of protein tyrosine phosphatases PTP1B, CD45, and LAR by peroxynitrite. Arch Biochem Biophys 369, 197-207.
Toker, A., and Newton, A. C. (2000a). Cellular signaling: pivoting around PDK-1. Cell 103, 185-188.
Toker, A. a. N., A. C. (2000b). AKT/protein kinase B is regulated by autophosphorylation at the hypothetical PDK-2 site. J Biol Chem 275, 8271-8274.
Tordjman, K. M., Leingang, K. A., James, D. E., and Mueckler, M. M. (1989). Differential regulation of two distinct glucose transporter species expressed in 3T3-L1 adipocytes: effect of chronic insulin and tolbutamide treatment. Proc Natl Acad Sci USA 86, 7761-7765.
Ullrich, A., Bell, J. R., Chen, E. Y., Herrera, R., Petruzzelli, L. M., Dull, T. J., Gray, A., Coussens, L., Liao, Y. C., Tsubokawa, M., et al., (1985). Human insulin receptor and its relationship to the tyrosine kinase family of oncogenes. Nature 313, 756-761.
Vanhaesebroeck, B., and Alessi, D. R. (2000). The P13K-PDK1 connection: more than just a road to PKB. Biochem J 346, 561-576.
Watson, R. T., Shigematsu, S., Chiang, S.-H., Mora, S., Kanzaki, M., Macara, I. G., Saltiel, A. R., and Pessin, J. E. (2001) Lipid raft microdomain compartmentalization of TC10 is required for insulin signaling and Glut4 translocation. J. Cell. Biol. 154, 829-840.
White, M. F. (1998). The insulin-signalling system: A network of docking proteins that mediate insulin action. Mol Cell Biochem 182, 3-11.
White, M. F., and Kahn, C. R. (1994). The insulin signaling system. J Biol Chem 269, 1-4.
Williams, M. R., Arthur, J. S. C., Balendran, A., van der Kaay, J., Poli, V., Cohen, P., and Alessi, D. R. (2000). The role of 3-phosphoinositide-dependent protein kinase 1 in activating AGC kinases defined in embryonic stem cells. Curr Biol 10, 439-448.

### SUMMARY OF THE INVENTION

The inventors have discovered that a key component of the PI-3 kinase-dependent insulin signaling pathway, namely PDK2, the putative kinase responsible for phosphorylating protein kinase B (“Akt”) on Ser473, is a membrane-associated kinas completely distinct from PDK1, the kinase that phosphorylates Akt on Thr308. This PDK2 activity can be separated from the bulk plasma membrane fraction in a solution containing a high chloride concentration (i.e., ≧100 mM Cl⁻). The inventors describe an in vitro assay reconstituting key aspects of PI-3 kinase-dependent insulin signaling derived from insulin-responsive cell components.

In the practice of this invention, an insulin-responsive cell, such as a muscle cell, adipocyt, islet cell or liver cell, is lysed and homogenized and its components separated into a plasma membrane fraction (“PM”), a low-density membrane fraction (“LDM”), which is enriched in endosomes, the Golgi apparatus, and insulin-responsive Glut4-containg vesicles, and a cytoplasmic fraction (“CYT”). The CYT comprises an insulin receptor substrate called Gab1, PDK1, Akt1 and Akt2 (isoforms of protein kinase B), and p85 component of PI 3-kinase. The LDM comprises insulin receptor substrate-1 and -2 (“IRS-1” and “IRS-2”), the p85 component of PI 3-kinase, and PDK2 activity. The PM comprises an insulin receptor, p85 component of PI 3-kinase, and PDK2 activity.

Based upon the discovery that an enzyme or catalyst having PDK2 activity resides within a membrane component of cells, wherein the membrane may be a plasma membrane or LDM component, the invention is drawn to a composition comprising components sufficient to reconstitute in vitro the early events in insulin signaling culminating in the phosphorylation of glucose synthase kinase-3 (“GSK3”) phosphorylation. The invention is also drawn (i) to methods of activating protein kinase B by facilitating the phophorylation of a serine that correlates to serine 473 of SEQ ID NO:1, and (ii) to methods of identifying agents that modulate insulin activity.

Preferably, these methods comprise the steps of (a) treating an insulin-responsive cell with insulin, (b) lysing and homogenizing the cell, (c) preparing PM, LDM and CYT fractions, (d) combining the CYT fraction with the LDM and/or PM fraction in a buffer comprising adenosine triphosphate (“ATP”) and less than 145 mM chloride (“low chloride”). Alternatively, the cell may not be treated with insulin. In the case of no insulin, P13-kinase is not activated and phosphatidylinositol(3,4,5)P₃ (“PIP3”) is not generated, therefore PIP3 or another phosphatidylinositiol phosphate compound, such as phosphatidylinositol(3,4)P₃ (“PI(3,4)P2”) may be added to the CYT/membrane/ATP low chloride mixture (“the assay mixture”).

In a method to identify agents that modulate insulin activity, an agent is added into the assay mixture. Any change in phosphorylation of protein kinase B or GSK3, or any change in glycogen synthase activity, relative to the assay mixture without an added agent, indicates that the agent modulates insulin activity.

### BRIEF DESCRIPTION OF THE DRAWINGS

FIG. 1. Reconstitution of early insulin signaling events in a cell-free assay. A. Scheme of the in vitro assay. B. Subcellular distribution of insulin signaling molecules. Fully differentiated 3T3-L1 adipocytes were fractionated by differential centrifugation as described in “Experimental Procedures.” PM, LDM, and CYT fractions were separated by SDS-PAGE (50 μg of protein), and analyzed by western blot analyses using each of the designated primary antibodies. PM(+ins) and PM(−ins) denote plasma membrane fractions that were derived from cells pre-exposed or not to insulin on ice prior to th fractionation.

FIG. 2. Optimization of the conditions used in the cell-free assay. The in vitro reactions were allowed to proceed for 15 min at 37° C. Immunoblots of the in vitro reactions were performed using phospho-AKT specific antibodies recognizing Thr308 and Ser473 respectively or phospho-GSK-3 specific antibodies that can detect GSK-3 phosphorylation on Ser21 (for the a isoform) and Ser9 (for the β isoform). The reactions were performed in the absence or presence of 150 mM sodium orthovanadate (VAN) and/or 1 μM microcystin-LR (MC). 1 mM DTT was also included during the subcellular fractionation steps and the subsequent in vitro reactions for several of the assays (lower two rows). INSULIN (±) refers to whether the PM used in the reactions was derived from cells pretreated or not on ice with insulin as herein described.

FIG. 3. Time course for insulin-dependent phosphorylation events. In vitro reactions containing PM and CYT were incubated at 37° C. for varying periods of time. INSULIN (±) refers to whether the PM used in the reactions was derived from cells pretreated or not on ice with insulin as herein described. A. Reactions were quenched (with a buffer containing SDS, vanadate, NaF, and sodium pyrophosphate), and protein samples (50 μg) were directly subjected to SDS-PAGE and immunoblot analysis using a phosphotyrosine antibody. An ATP depleting system (“no ATP”) was added to certain reactions (last 4 lanes). B. In vitro reactions were quenched (with a buffer containing Triton X-100, 20 mM EDTA, vanadate, NaF, and sodium pyrophosphate), and either the insulin receptor (IR), IRS-1, or the p85 subunit of PI 3-kinase was immunoprecipitated. The resulting immunoprecipitates were subjected to immunoblot analysis using a phosphotyrosine antibody. Arrows denote the migration of IRS-2 and IRS-1 respectively.

FIG. 4. Time dependency and specificity of Akt and GSK-3 insulin-dependent phosphorylation. In vitro reactions containing PM and CYT were incubated at 37° C. for varying periods of time. INSULIN (±) refers to whether the PM used in the reactions was derived from cells pretreated or not on ice with insulin as herein described. A. Time course (0-15 minute) for Akt and GSK-3 phosphorylation. Samples were subjected to immunoblot analysis using phospho-Akt and phospho-GSK-3 antibodies. The GSK-3 antibody recognizes phosphorylation of Ser21 (for the a isoform) and Ser9 (for the β isoform). An ATP depleting system (“no ATP”) was added to certain reactions (last 4 lanes). B. In vitro reactions were allowed to proceed for 15 min in the absence or presence of 100 nM wortmannin (WT), and samples were subjected to immunoblot analysis using phospho-Akt and phospho-GSK-3 antibodies.

FIG. 5. Addition of soluble GST-insulin receptor fusion protein is insufficient for Akt activation in vitro. In vitro reactions containing PM and CYT were incubated at 37° C. for 10 min. INSULIN(±) refers to whether the PM used in the reactions was derived from cells pretreated or not on ice with insulin as described in “Experimental Procedures.” GST-IR (±) indicates the presence or absence of the GST-insulin receptor fusion protein. A. In vitro reactions were quenched (with a buffer containing Triton X-100, 20 mM EDTA, vanadate, NaF, and sodium pyrophosphate) and the samples were immunoprecipitated using an antibody recognizing the insulin receptor (IR). The resulting immunoprecipitates were subjected to immunoblot analysis using a phosphotyrosine antibody. B. Aliquots of the same samples were immunoprecipitated using an antibody recognizing IRS-1. The resulting immunoprecipitates were subjected to immunoblot analysis using a phosphotyrosine antibody. C. Aliquots of the same samples were immunoprecipitated using an antibody recognizing the p85 regulatory subunit of PI 3-kinase. The resulting immunoprecipitates were subjected to immunoblot analysis using a phosphotyrosine antibody. D. Aliquots of the same samples were subjected to immunoblot analysis using phospho-Akt antibodies.

FIG. 6. Soluble tyrosine-phosphorylated adaptor proteins are dispensable for Akt activation in vitro. In vitro reactions containing PM and CYT were incubated at 37° C. for 10 min. INSULIN(±) refers to whether the PM used in the reactions was derived from cells pretreated or not on ice with insulin. Immunodepleted CYT were prepared as described in “Experimental Procedures.” CYT-CON refers to control CYT mock-immunodepleted with an irrelevant antibody. A. The components of the in vitro reactions were subjected to immunoblot analysis using antibodies specific for IRS-1, IRS-2, or Gab1. B. In vitro reactions with the indicated componenents were subjected to immunoblot analysis using a phosphotyrosine antibody (PY20). C. In vitro reactions were subjected to immunoblot analysis using phospho-Akt antibodies.

FIG. 7. PDK1 and PDK2 activities can be segregated. In vitro reactions containing different combinations of PM, CYT, and LDM were incubated at 37° C. for 15 min. PM(SW) and LDM(SW) are PM and LDM that were salt-washed with 1 M NaCl respectively during the isolation procedure. “CYT-PDK1” refers to cytosol from which PDK1 had been immunodepleted. “CYT-CON” refers to a similarly treated control cytosol mock-immunodepleted with an antibody recognizing Glut1. A. The components of the in vitro reactions were subjected to immunoblot analysis using an antibody specific for PDK1. B. In vitro reactions were subjected to immunoblot analysis using phospho-Akt antibodies. INSULIN (±) refers to whether the PM used in the reactions was derived from cells pretreated or not on ice with insulin as herein described. C. 10 μM PIP3 was added or not to various 3T3-L1 adipocyte components isolated from non-insulin treated cells and incubated for 15 min at 37° C. in the presence of ATP. The reaction samples were subjected to immunoblot analysis using phospho-Akt antibodies. D. The components of the in vitro reactions were subjected to immunoblot analysis using an antibody specific for ILK.

FIG. 8. Chloride inhibits PDK1 and PDK2 enzymatic activity. PM and CYT were incubated for 15 min at 37° C. INSULIN (±) refers to whether the PM used in the reactions was derived from cells pretreated or not on ice with insulin as herein described. PIP3 (±) denotes whether 10 μM PIP3 was added or not to PM(−ins) and CYT. KGlu, KAc, KCl, NaCl refers to whether the reactions were carried out in the presence of 140 mM potassium glutamate/5 mM NaCl, 140 mM potassium acetate/5 mM NaCl, 140 mM potassium chloride/5 mM NaCl, or 145 mM sodium chloride respectively. The reaction samples were subjected to immunoblot analysis using phospho-Akt antibodies.

FIG. 9. Rescue of PDK2 activity in salt-extracted plasma membranes. In vitro reactions containing different subcellular components were incubated for 15 min at 37° C. PM(SW) are plasma membranes that were salt-washed in 1 M NaCl for 30 min and then recovered as a pellet by centrifugation at 37,000×g for 20 min. Ext-LoS refers to proteins found in the supernatant after the 37,000×g centrifugation step. The reaction samples were subjected to immunoblot analysis using phospho-Akt antibodies. A. INSULIN (±) refers to whether the PM used in the reactions was derived from cells pretreated or not on ice with insulin as herein described. B. PIP3 (±) denotes whether 10 μM PIP3 was added or not to PM(−ins) and CYT.

FIG. 10. PDK2 activity extracted from PM with high salt can be pelleted at high speed. In vitro reactions containing different subcellular components were incubated for 15 min at 37° C. PM(SW) are plasma membranes that were salt-washed in 1 M NaCl for 30 min and then recovered as a pellet by centrifugation at 37,000×g for 20 min. Ext-LoS refers to proteins found in the supematant after the 37,000×g centrifugation step. Centrifugation of Ext-LoS at 200,000×g for 1 h resulted in a pellet (Ext-HiP) and a supematant (Ext-LoS). PIP3 (±) denotes whether 10 μM PIP3 was added or not to the reaction. The reaction samples were subjected to immunoblot analysis using phospho-Akt antibodies. A. Various extracts (Ext-LoS, Ext-HiP, and Ext-HiS) were tested for PDK2 activity in a PIP3-stimulated reaction containing PM(SW) and CYT. B. Various extracts (Ext-LoS, Ext-HiP, and Ext-HiS) were tested for PDK2 activity in a PIP3-stimulated reaction containing CYT alone.

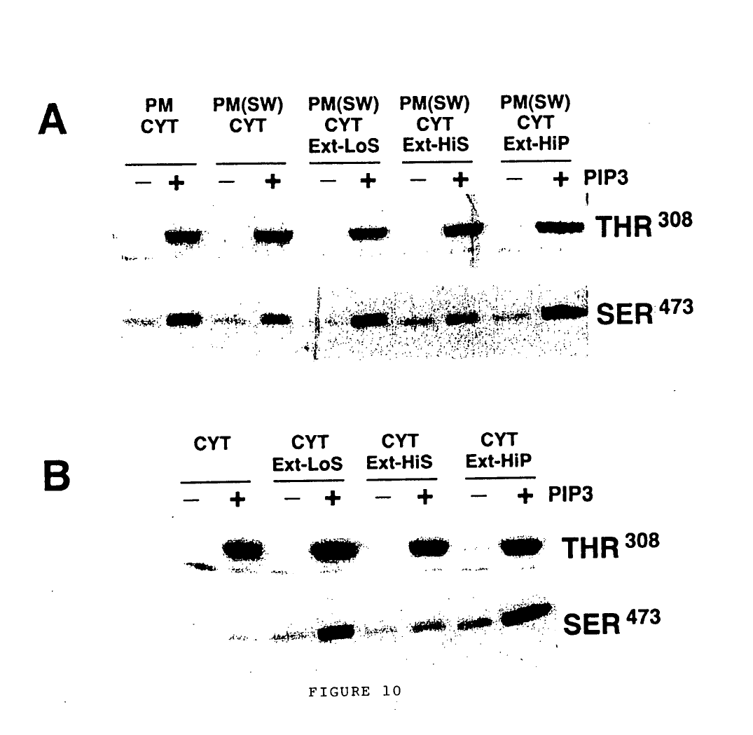

FIG. 11. PDK2 activity in the PM colocalizes with focal adhesions. 50 μg of PM, PM(SW), and Ext-HiP were compared by immunoblot analysis using antibodies directed against several cytoskeletal and PM associated proteins.

### DETAILED DESCRIPTION OF THE INVENTION

### 1. DEFINITIONS

The term “phosphorylation” means the addition of a phosphate group to an amino acid, usually a serine, threonine or tyrosine.

The term “insulin-responsive cell” means any vertebrate cell that naturally expresses an insulin receptor and activates glycogen synthase (“GS”), which catalyses the formation of glycogen from glucose monomers, in response to insulin exposure. Examples of insulin-responsive cells include muscle cells, liver cells, adipocytes and islet cells.

The term “protein kinase B” means an enzyme that is activated via phosphatidylinositol lipids such as PIP3 or PI(3,4)P2 and is capable of phosphorylating glycogen synthase kinase-3 (“GSK3”). Protein kinase B (“PKB” or “Akt”) denotes multiple enzymes, including v-Akt, PKBα (also known as Akt1 and which human sequence is depicted in SEQ ID NO:1), PKBβ (also known as Akt2), PKBγ also known as Akt3), and drosophila protein kinase B (“DRKB”). PKBα, as set forth in SEQ ID NO:1, is used herein as a prototype for all protein kinase B. Threonine 308 (T³⁰⁸) and serine 473 (S⁴⁷³) of PKBα correspond to T⁵⁸³ and S⁷⁴⁸ of v-Akt, T³⁰⁹ and S⁴⁷⁴ of PKBβ and T³⁴² and S⁵⁰⁵ of DPKB, respectively. PKBγ does not have an analogous serine at the C-terminus corresponding to S⁴⁷³. Protein kinase B, and isoforms thereof, are reviewed in Coffer et al. (1998), which is herein incorporated by reference.

The phrase “modulates insulin activity” or “modulate insulin activity” means to significantly increase or decrease the level of phosphorylation of protein kinase B or GSK3, or to increase or decrease the activity of glycogen synthase, relative to the baseline level of protein kinase B or GSK3 phosphorylation, or glycogen synthase activity. A significant change is at least ±0.5% in the level of glycogen synthase activity or in the mole ratio of phophorylated amino acids, with a p≦0.05. The baseline level of phosphorylation will be determined by the negative control of the assay, which is the execution of the assay in the absence of an agent. The level of phosphorylation may be determined by any means known in the art, including immunoblotting. “Assay” as used herein means combining an “assay mixture” (defined as reconstituted insulin-responsive cell-extracts, which includes CYT, LDM and/or PM, and ATP in low chloride buffer) with an agent and determining the level of phosphorylation of protein kinase B or GSK3, or glycogen synthase activity.

An “agent” may be any salt, ion, compound, chemical, chemical library, atom, buffer, metal, temperature change, pH condition, radionuclide, peptide, protein, nucleic acid, carbohydrate, lipid, microbe, virus, cell, adduct or moiety.

The term “membrane fraction” means any phospholipid bilayer, which comprises integral membrane proteins, other lipid soluble compounds, cytoskeleton, and salt-extractable membrane associated proteins (as is commonly known in the art). As used in the practice of this invention, a “membrane fraction” is obtained from an insulin-responsive cell. Preferably, the insulin-responsive cell has been treated with an effective amount of insulin prior to cell lysis or homogenization. Membrane fractions include the plasma membrane fraction (“PM”) and the low-density membrane fraction (“LDM”), as are known in the art. There are two types of PM used in the practice of this invention, PM(−ins) (plasma membrane fraction derived from a cell that has not been treated with an effective amount of insulin) and PM(+ins) (plasma membrane fraction derived from a cell that has been treated with an effective amount of insulin). When a PM(−ins) fraction is employed in the practice of this invention, a phosphatidylinositiol phosphate molecule, preferably PIP3 or PI(3,4)P2, must be added to the assay mixture. PM and LDM each comprise a PDK2 activity. The PDK2 activity may be salt-extracted from the bulk PM.

The phrase “low chloride” denotes a chloride concentration of an aqueous solution that is permissible for PDK2 activity and maintains the PDK2 activity in a PM fraction. As used herein, “low chloride” means a chloride concentration of below 145 mM.

The term “desalt” or “process of desalting” means the reduction of the ionic strength of an aqueous solution usually comprising a protein or some other macromolecule of interest, such as a lipid or carbohydrate. Myriad methods are available in the art to effect desalting, such as dialysis and molecular sieve chromatography (i.e., desalting columns). As used herein, the term “desalt” applies to the reduction of the chloride concentration to below 145 mM of a solution comprising a PDK2 activity, which was salt-extracted from the bulk PM fraction.

The term “salt-extracted” denotes the process of treating a membrane fraction with a high salt solution, usually to remove membrane associated proteins. As used herein, the term “salt-extracted” refers to the process of treating a PM fraction with a solution comprising ≧145 mM chloride, preferably 1M NaCl, to extract PDK2 activity. A “salt-extracted aqueous phase” is the aqueous phase that remains after salt extraction, whereas a “salt-extracted membrane fraction” is the membrane phase that remains after salt-extraction.

The phrase “desalted aqueous fraction” refers to a salt-extract aqueous phase that has undergone the process of desalting. As used herein, “desalted aqueous fraction” denotes an aqueous solution comprising a PDK2 activity in a solution comprising less than 145 mM chloride.

The “cytoplasmic fraction” or “CYT” denotes that portion of a cell homogenate that is free of plasma membrane or low density membrane fractions. As used herein, the “cytoplasmic fraction” comprises an insulin receptor substrate called Gab1, PDK1, Akt1 and Akt2 (isoforms of protein kinase B), and a p85 component of PI 3-kinase.

“PDK1” or phosphatidylinositol phosphate dependent kinase-1 is an enzyme found in the cytoplasmic fraction of insulin-responsive cells. PDK1 catalyzes the transfer of a phosphate group from ATP to threonine 308 of PKBα (or threonine 309 of PKBβ).

PDK2 activity” or “PDK2” or phosphatidylinositol phosphate dependent kinase-2 is an enzyme found in the PM and LDM fractions of insulin-responsive cells. PDK2 activity may be extracted from the bulk PM fraction under high salt conditions. PDK2 catalyses the transfer of a phosphate group from ATP to serine 473 of PKBα (or serine 474 of PKBβ).

“Phosphatidylinositiol phosphate molecule” is a secondary messenger molecule derived from the phosphorylation of phosphatidylinositol(4,5)P₂ (“PI(4,5)P₂”) by phosphatidylinositol-3 kinase (“PI3K”). Preferred “phosphatidylinositiol phosphate molecules” stimulate the activation of protein kinase B and include phosphatidylinositol(3,4,5)P₃ (“PIP3”) and phosphatidylinositol(3,4)P₂ (“PI(3,4)P₂″). Phosphatidylinositiol phosphate molecules may be used in the assay mixture when PM(−ins) fractions are used.

An “effective amount of insulin” is any amount of insulin that effectively activates P13K activity in an insulin-responsive cell. Preferably, an effective amount of insulin is greater than 10 nM of insulin.

### 2. OVERVIEW OF THE INVENTION

The basis for this invention resides in the discovery by the inventors of a novel PDK2 activity in a membrane fraction an insulin-responsive cell. As described herein, that PDK2 activity may be salt-extracted from bulk plasma membrane fraction. This discovery has enabled the inventors to develop an in vitro reconstituted system of the insulin-signaling pathway (“assay mixture”), comprising a cytoplasmic fraction and a fraction containing the PDK2 activity. In one embodiment of the invention, the in vitro reconstituted system allows for the in vitro phosphorylation of protein kinase B on serine 473. In another embodiment, the in vitro reconstituted system allows for the in vitro activation of protein kinase B and subsequent phosphorylation of GSK3. In another embodiment, the in vitro reconstituted system comprises an assay platform for the identification or discovery of agents that modulate or influence the PI3K-mediated insulin signaling. An agent or library of agents may be added to the assay mixture and the phosphorylation state of any protein or phosphatidylinositide may be determined by common art recognized means, such as immunoblotting. Alternatively, the activity of enzymes in the insulin response pathway may be measured, such as glycogen synthase. Agents discovered through the practice of this invention may have utility in the treatment of diseases of energy metabolism, such as diabetes and obesity. Methods of determining the phosphorylation status of known proteins is well known in the art. Methods of determining enzyme activity of known proteins (such as GSK3 and glycogen synthase) are also well known in the art.

The following examples are intended to illustrate but not limit the invention. While they are typical of those procedures that might be used, other procedures known to those skilled in the art may alternatively be used in the practice of this invention. The spirit and scope of the invention is not limited by the following examples, but rather by the claims that follow.

### 3. EXAMPLES

### General Methodology

Cell culture of 3T3L1 adipocytes—3T3-L1 preadipocytes obtained from the American Type Culture Collection were grown to confluence and 48 hours later subjected to differentiation as described previously (Tordjman, 1989). 3T3-L1 adipocytes were used 10 to 14 days after initiating differentiation.

Isolation of subcellular components—Mature 3T3-L1 adipocytes grown on 10-cm dishes were serum-starved overnight. The cells were then rapidly washed three times with ice-cold serum-free DMEM, and maintained further for 15 minutes at 4° C. in serum-free DMEM in the absence or presence of 1 μM insulin. Cells were then washed three times with ice-cold PBS, scraped in 2 ml/dish of ice-cold HES buffer (50 mM Hepes, pH 7.4, 255 mM sucrose, and 1 mM EDTA) containing protease inhibitors (0.082 TIU/ml aprotinin (Sigma), 0.1 μg/ml leupeptin, 0.1 μg/ml antipain, 0.5 μg/m trypsin inhibitor, 0.1 μg/ml chymostatin, 0.1 μg/ml pepstatin A, and 0.5 mM phenylmethylsulfonyl fluoride) and then homogenized at 4° C. by passing the cells 10 times through a Yamato SC homogenizer at a speed of 1200 rpm. The PM fraction was obtained by differential centrifugation and sucrose cushion flotation as described previously (Piper, 1991), and designated as either ‘PM(−ins)’ or ‘PM(+ins)’ according to whether the starting cell source was exposed insulin. The LDM fraction was obtained from basal cells as described previously (Piper, 1991). PM and LDM, subsequent to their isolation, were resuspended in IC buffer (20 mM Hepes, pH 7.4, 140 mM potassium glutamate, 5 mM NaCl, 1 mM EGTA, and protease inhibitors). A highly concentrated CYT fraction was prepared by washing the 3T3-L1 adipocytes three times with ice-cold IC buffer, then removing the buffer as much as possible by aspiration, followed by cell scraping and homogenizing with a ball-bearing homogenizer. The supernatant was recovered following an ultracentrifuge spin for 1 hour at 200,000×g. For the preparation of PM salt-extracted proteins, plasma membranes pelleted after sucrose cushion flotation were resuspended in 200 μl of IC buffer containing 1 M NaCl, incubated on ice for 30 min, and then subjected to centrifugation in a TLA-100.3 fixed angle rotor for 20 min at 37,000×g. The pellet formed from this spin (PM(SW)) was resuspended in IC buffer. The 1 M NaCl was removed from the supematant (Ext-LoS) using a 1 ml Sephadex G-25 spin column that was pre-equilibrated with 2 ml of IC buffer. Equilibrated columns were centrifuged for 1 min at 1000 rpm prior to applying the sample to the top of the resin. Centrifugation of the sample through the spin column for 1 min at 1000 rpm removed the 1 M NaCl. For certain experiments, Ext-LoS was further centrifuged in a TLA-100.3 fixed angle rotor at 200,000×g for 1 h to produce a supernatant (Ext-HiS) and a pellet (Ext-HiP). Ext-HiP was resuspended in IC buffer and Ext-HiS was desalted with a 1 ml Sephadex G-25 spin column.

In vitro assay—Samples were prepared on ice by mixing in various combinations LDM (˜2.5 mg/ml final concentration), CYT (˜3 mg/ml final concentration), and PM(±ins) (˜0.5 mg/ml final concentration). Reaction volumes, ranging from 100-200 μl, were adjusted as necessary with IC buffer. Reactions were initiated with the addition of either an ATP regenerating system (final reaction concentrations: 1 mM ATP, 8 mM creatine phosphate, 30 units/ml creatine phosphokinase, and 5 mM MgCl₂) or an ATP depleting system (final reaction concentrations: 25 units/ml hexokinase and 5 mM glucose). Samples were incubated with rotation at 37° C. for 0-15 minutes. The reactions were quenched by addition of an equal volume of buffer B (50 mM Hepes, pH 7.4, 150 mM NaCl, 2 mM sodium vanadate, 100 mM NaF, and 10 mM sodium pyrophosphate) either containing 2% SDS and 1 mM EDTA (for samples to be run directly on SDS-PAGE) or 2% Triton X-100 and 40 mM EDTA (for samples to be immunoprecipitated). For certain in vitro reactions, as indicated, some of the following were also added (final concentrations): (1) 1 mM DTT; (2) 150 μM sodium vanadate; (3) 1 μM microcystin LR (Calbiochem); (4) 100 nM wortmannin (Calbiochem); (5) 1 μg/100 μl reaction volume of recombinant human insulin receptor β subunit-GST fusion protein (Calbiochem; 407697); (6) 10 μM phosphatidylinositol(3,4,5)P3 (PIP3; Calbiochem) in a sonication mixture of 100 μM phosphatidylcholine (Avanti Polar Lipids) and 100 μM phosphatidylserine (Avanti Polar Lipids). For the preparation of PDK1-immunodepleted CYT, pre-cleared CYT was incubated for 1.5 hours at 4° C. with protein G-agarose (Upstate Biotechnology) bound with anti-PDK1 polyclonal sheep IgG (Upstate Biotechnology catalog no. 06637; 5 μg of IgG/mg of CYT).

Immunoblot analysis and immunoprecipitation—Protein samples from the in vitro assay were subjected to SDS-PAGE and transferred to nitrocellulose. Phospho-specific antibodies recognizing the phosphorylated forms of Akt or GSK-3 were obtained from New England Biolabs. The monoclonal anti-phosphotyrosine antibody PY20 and antibodies directed against ILK, paxillin, and integrin β1 receptor were purchased from PharMingen. PDK1 antibody used for immunoblot analysis was purchased from Upstate Biotechnology (catalog no. 06-906) as well as vinculin, insulin receptor, IRS-2, Gab1, AKT-(1-3), and caveolin antibodies. Actin antibody was from Chemicon. The arp3 antibody was a kind gift of Dr. John Cooper in the Cell Biology and Physiology Department at Washington University. Immunoprecipitation of IRS1 was accomplished by use of a polyclonal rabbit antibody raised against the carboxy-terminal 14 amino acids of rat IRS-1. Immunoprecipitation of the insulin receptor, Gab1, and the p85 subunit of PI-3 kinase were carried out by use of the appropriate antibody purchased from Upstate Biotechnology.

### Example 1:

### Characterization of the Cell-Free Assay System (“Assay Mixture”)

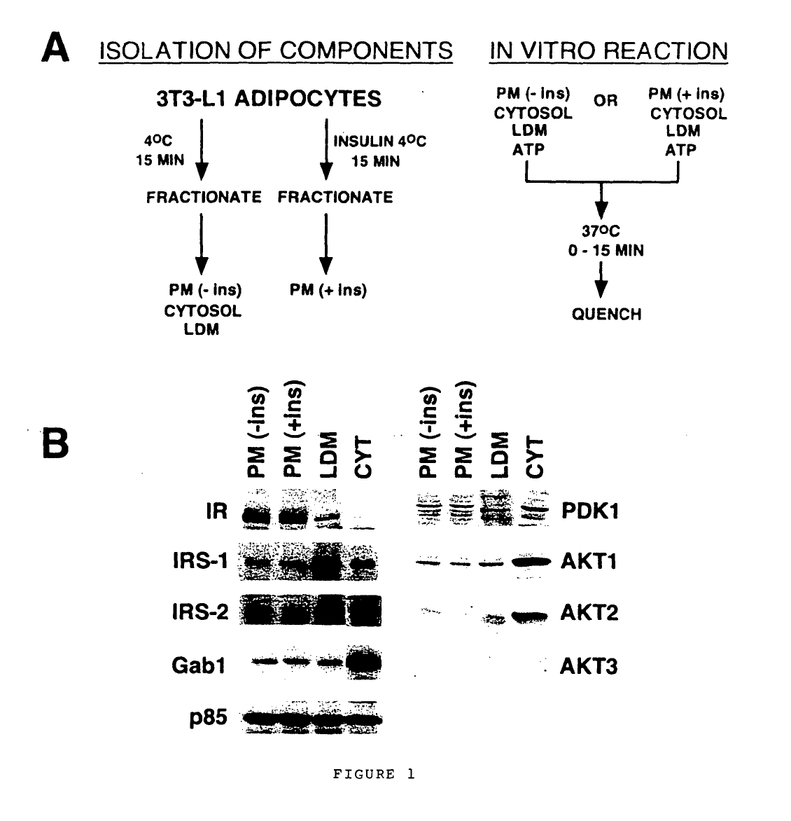

Key aspects of the insulin-signaling pathway have been reconstituted using subcellular fractions of 3T3-L1 adipocytes, the “assay mixture”. Adipocytes typically exhibit a ˜10-20 fold increase in glucose uptake in response to acute stimulation with insulin (Calderhead, 1990). The capacity to respond to this extent is acquired during the course of adipocyte differentiation, during which the expression levels of signaling components (such as the insulin receptor and IRS-1) (Reed, 1977; Rice, 1992; Rubin, 1977) and effector molecules (such as the insulin-responsive glucose transporter Glut4) (James, 1989; Tordjman, 1989) are dramatically induced. Extensively characterized subcellular fractionation protocols exist for adipocytes, allowing the reproducible recovery of distinct subcellular components with relative ease (Jarett, 1974; Piper, 1991; Simpson, 1983). The premise of the basic in vitro assay is diagrammed in FIG. 1A. Fully differentiated 3T3-L1 adipocytes in the basal state were first cooled rapidly by washing with ice-cold buffer, and then maintained at 4° C. in the presence or absence of 1 μM insulin. The cold temperature incubation allowed insulin to bind its cell surface receptor but prohibited subsequent intracellular signaling. Following the cold temperature incubation, purified PM fractions were obtained by differential centrifugation and sucrose cushion flotation (referred to as ‘PM(−ins)’ or ‘PM(+ins)’ according to whether the cell source was exposed to insulin). Basal cells were also used to obtain the LDM and cytosol (CYT). The LDM fraction is enriched in endosomes, the Golgi apparatus, and insulin-responsive Glut4-containg vesicles, as well as insulin-signaling molecules such as IRS-1 and PI 3-kinase (Clark, 1998). To initiate the in vitro assay, the 3T3-L1 subcellular fractions (PM(−ins) or PM(+ins); LDM; CYT) were mixed in various combinations in the presence of an ATP regenerating system and incubated at 37° C. for up to 15 minutes, thereby allowing insulin (carried through to this point, in reactions containing PM(+ins), via high-affinity interaction with its receptor) to exert its effects. The concentrations of PM, LDM, and CYT protein in a typical reaction were 0.5, 2.5, and 3 mg/ml respectively.

The starting subcellular fractions were examined for the presence of insulin signaling molecules by immunoblot analysis (FIG. 1B). The insulin receptor was highly enriched in the PM fraction and the amount did not vary with exposure to insulin. IRS-1 and IRS-2 were found mainly in the LDM and, to a lesser extent, the CYT fraction as previously reported (Inoue, 1998). IRS-3, which is present in primary adipocytes, is not expressed in 3T3-L1 adipocytes (Lavan, 1993). In contrast to the IRS proteins, Gab1 (Holgado-Madruga, 1996), another insulin receptor substrate, was found exclusively in the cytosol. The p85 subunit of PI 3-kinase was present to a significant degree in all three subcellular fractions. PDK1 was mainly found in the cytosol. Akt1 and Akt2 were found almost completely in the cytosol whereas Akt3 was not expressed in these cells.

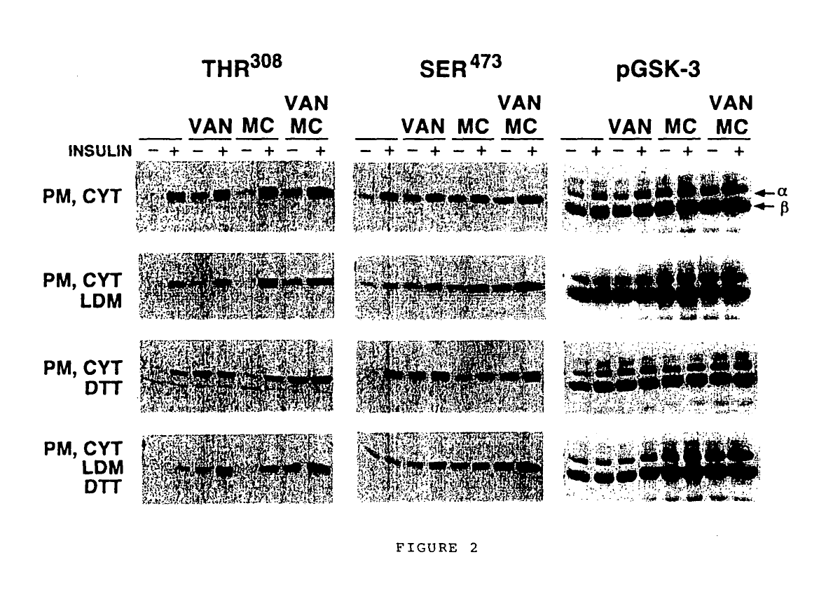

It is well known in the art that the generation of PIP3 by PI-3 kinase leads to the activation of Akt by phosphorylation of two of its residues—Thr308 and Ser473 (Alessi, 1996) (Akt1 nomenclature). Akt, in turn, can phosphorylate glycogen synthase kinase-3 (GSK-3)on Ser21 (for the α isoform) or Ser9 (for the β isoform) (Cross, 1995). The phosphorylation status of Akt and GSK-3 in the instant in vitro system was examined by immunoblot analysis using appropriate phospho-specific antibodies. The phospho-specific Akt antibodies used in this study (New England Biolabs) are capable of detecting both Akt1 (PKBα) and Akt2 (PKBβ) phosphorylation (Hill, 1999), although Akt2 has been reported to be the major isoform in 3T3-L1 adipocytes (Hill, 1999; Summers, 1999). The in vitro reactions were performed in the absence or presence of phosphatase inhibitors. As shown in FIG. 2, Akt was properly phosphorylated in vitro on both Thr308 and Ser473 in response to insulin in reactions containing PM(+ins). The occurrence of insulin-dependent signaling suggested that the cytoplasmic domain of the insulin receptor (which contains the intrinsic tyrosine kinase) was properly oriented with respect to the membrane in order to access its substrates. Using alkaline phosphatase activity as the ecto-domain marker (Garen, 1960; Schlemmer, 1992), it was demonstrated that 70% of the prepared PM vesicles were oriented inside-out. Moreover, the PM vesicles were apparently sealed because Akt was not activated by direct addition of insulin to in vitro reactions containing PM(−ins). Despite being enriched in signaling molecules such as PI-3 kinase and IRS-1/2 (Clark, 1998), the LDM fraction appeared to be dispensable for the phosphorylation of Akt. In fact, a stronger insulin-stimulated signal was consistently observed for both Akt phosphorylation sites in reactions excluding the LDM in the absence of the broad specificity Ser/Thr phosphatas inhibitor microcystin-LR. This suggested that LDM might contain a phosphatase activity capable of acting on Akt. Generally, the tyrosine phosphatase inhibitor vanadate appeared to elevate the signal for both Akt phosphorylation sites in reactions containing PM(+ins); however, at the same time, the corresponding control (basal) signal in reactions containing PM(−ins) was also elevated, thus blunting the discernable insulin response. Microcystin also increased the insulin-stimulated signal for both Akt phosphorylation sites; however, this phosphatase inhibitor elevated the basal signal only for the Ser473 site. Some of the in vitro reactions were also performed in the presence of 1 mM DTT, which was included in order to mimic the reducing environment found inside cells (FIG. 2; lower two rows). DTT appeared to inhibit the phospho-Akt signal in most cases. There are at least two reasons to explain why DTT failed to facilitate signaling. First, a reducing environment, such as that provided by DTT, is required for optimal activity of certain tyrosine phosphatases, which can be expected to downregulate insulin signaling (Denu, 1998; Takakura, 1999). Second, DTT is known to inhibit the phosphatase-countering activity of vanadate (Gordon, 1991). The insulin-stimulated phosphorylation of GSK-3 (α/β on Ser (21/9) mirrored that of Akt (FIG. 2). The empirical comparison of various conditions demonstrated that the reaction conditions containing PM and CYT but excluding DTT, vanadate, and microcystin, stimulated the greatest fold difference in insulin-responsive phosphorylation of Akt and GSK-3 between basal cells and the insulin-stimulated cells. Under these conditions both phosphorylation and dephosphorylation reactions could occur since phosphatase inhibitors were not necessary for detecting insulin-stimulated Akt phosphorylation.

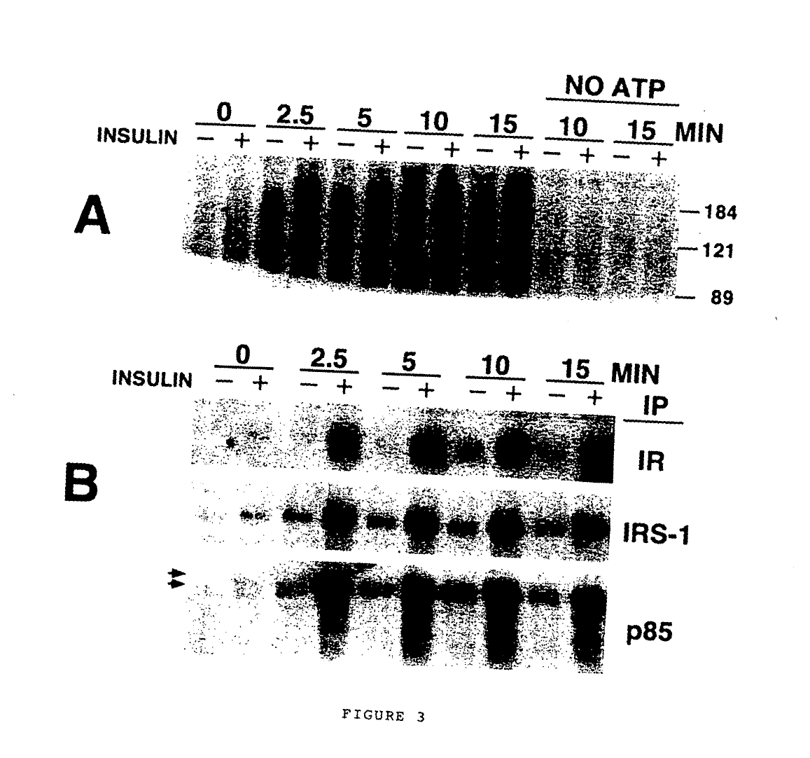

The time course for insulin receptor-mediated tyrosine phosphorylation under the optimal conditions described above was followed by immunoblot analysis using an anti-phosphotyrosine antibody. Two bands at ˜160 kDa and ˜95 kDa appeared in response to insulin, corresponding to the molecular mass of IRS-1/2 and the β subunit of the insulin receptor, respectively (FIG. 3A). Phosphotyrosine signals were completely absent when ATP was depleted from the reaction (FIG. 3A, last 4 lanes), thus ruling out the possibility of significant reaction contamination by intact cells. The identities of the two insulin-dependent phosphotyrosine bands were confirmed by solubilizing the reaction with 1% Triton X-100 and then immunoprecipitating with an antibody recognizing either the β subunit of the insulin receptor or IRS-1 (FIG. 3B). For both of these proteins, the phosphotyrosine signal peaked at 2.5 minutes from the start of the reaction and somewhat decreased thereafter, probably due to dephosphorylation.

The in vitro recruitment of PI-3 kinase to tyrosine-phosphorylated adaptor proteins was also examined. After solubilizing the reaction mixture with 1% Triton X-100, tyrosine-phosphorylated proteins capable of co-immunoprecipitating with the p85 subunit of PI-3 kinase were detected by immunoblot analysis (FIG. 3B, bottom panel). Insulin stimulated the association of PI-3 kinase with a tyrosine-phosphorylated protein doublet corresponding to the molecular mass of IRS-1 and IRS-2, mimicking what occurs in vivo (Inoue, 1998; Kelly, 1993). A minor population of PI-3 kinase was found to be complexed with a protein of ˜95 kDa, which may be the autophosphorylated β subunit of the insulin receptor. It is important to note that others have observed the in vivo association between PI-3 kinase and the activated insulin receptor (Endemann, 1990; Ruderman, 1990).

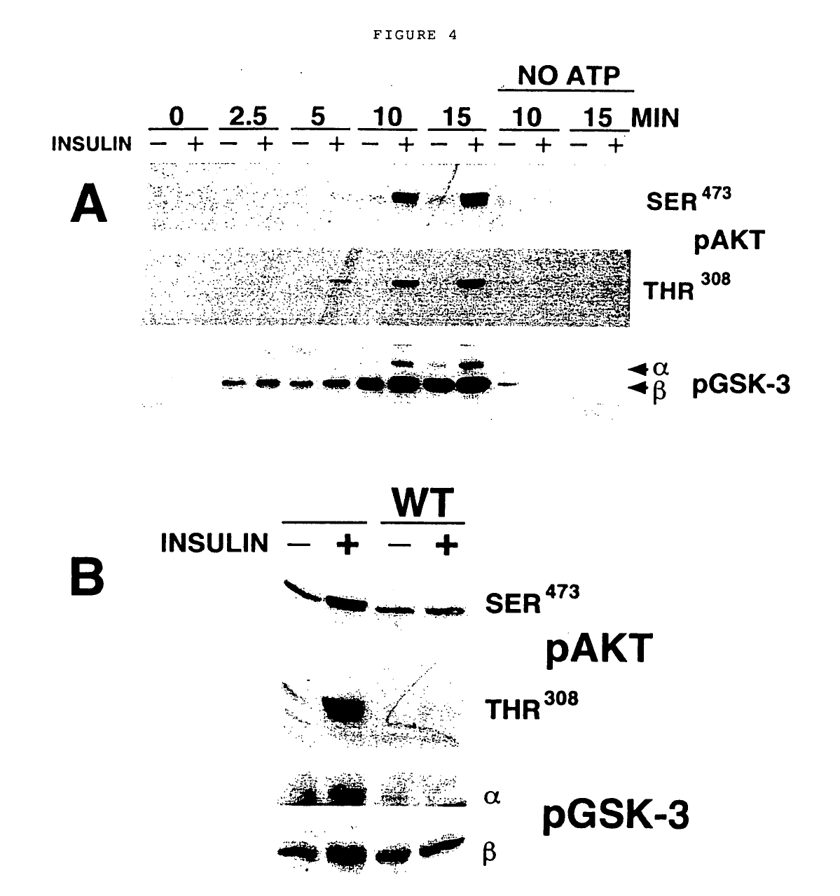

The time course for the phosphorylation of Akt was assessed by immunoblot analysis using phospho-Akt specific antibodies (FIG. 4A). Akt phosphorylation exhibited kinetics delayed relative to that of IRS-1 tyrosine phosphorylation, with the insulin-stimulated signal peaking at approximately 10 to 15 minutes from the start of the reaction. The time course for GSK-3 phosphorylation was similar to that of Akt (FIG. 4A). As was observed for tyrosine phosphorylation, the phosphorylation of Akt and GSK-3 in vitro was completely dependent on exogenous ATP (FIG. 4A, last 4 lanes). Also, the addition of 100 nM wortmannin to the reaction completely abrogated insulin-stimulated Akt phosphorylation on Thr308 and Ser473 and GSK-3(α/β phosphorylation (FIG. 4B); this indicated that the in vitro kinase activities targeting both Akt sites and the subsequent GSK phosphorylation were PI-3 kinase-dependent, mimicking in vivo characteristics (Alessi, 1996).

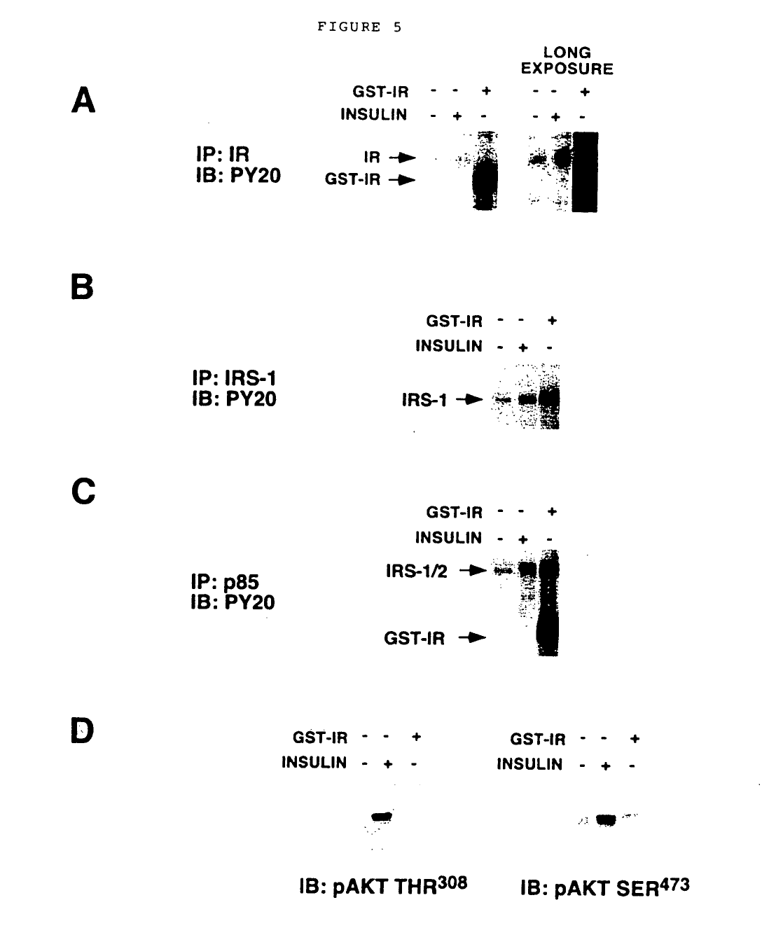

The preceding data demonstrated that early insulin signaling events dependent on PI 3-kinase, up to and including Akt and GSK-3 phosphorylation, appeared to be faithfully reconstituted with reasonable efficiency in our in vitro system. A cell-free system offers several advantages in answering questions concerning insulin action that would be extremely difficult or impossible to address in a satisfactory manner in an intact cell. In particular, facile experimental access to all components of our system allows manipulations such as the introduction of membrane-impermeable reagents or the depletion of cellular factors. As a demonstration of this principle, we added a soluble recombinant insulin receptor kinase domain fusion protein (derived from the catalytic β subunit) to an in vitro reaction containing PM(−ins) and CYT. The fusion protein was robustly tyrosine-phosphorylated in the absence of insulin, reflective of its constitutive activity (FIG. 5A). The signal derived from the insulin receptor fusion protein (72 kDa) was in vast excess relative to that derived from the native insulin receptor β subunit (95 kDa) in a parallel reaction containing PM(+ins) and CYT. The insulin receptor fusion protein was capable of phosphorylating IRS-1 (FIG. 5B). The level of tyrosine-phosphorylated IRS-1 was considerably greater in the reaction containing the insulin receptor fusion protein as compared to that of the reaction in which in vitro signaling was initiated by insulin according to our basic protocol. IRS-1/2 phosphorylated by the insulin receptor fusion protein was found in a complex with the p85 regulatory subunit of PI 3-kinase as demonstrated by co-immunoprecipitation (FIG. 5C), thereby establishing that the insulin receptor fusion protein was indeed acting on physiologically relevant sites of substrate molecules. The tyrosine-phosphorylated insulin receptor fusion protein was also associated with a large amount of p85 (FIG. 5C), the favorable interaction most likely driven by mass action (as mentioned earlier, p85 is capable of binding to the activated insulin receptor). Despite the successful propagation of these early steps of insulin signaling involving tyrosine phosphorylation, the addition of the insulin receptor fusion protein failed to activate downstream Akt-neither Thr308 nor Ser473 was phosphorylated (FIG. 5D). In a parallel reaction containing PM(+ins) and CYT, Akt was efficiently phosphorylated at both regulatory sites.

The preceding data suggest that the signal from the insulin receptor must originate at the plasma membrane in order for Akt to be activated efficiently. The activated insulin receptor in soluble form can phosphorylate its physiological substrates, but the resulting signaling complexes, despite being present in abundant absolute levels, are likely to be mislocalized and incapable of stimulating further downstream signaling.

The most probable impediment to signaling initiated by the insulin receptor fusion protein is at the level of PIP3 generation. Under these circumstances, the activated PI 3-kinase in complex with IRS proteins may have limited access to its phosphoinositide substrate present in the inner leaflet of the plasma membrane lipid bilayer. Random diffusional intermolecular encounters lead to inefficient signal transduction. In vivo, the signaling components are likely to be spatially segregated in such a way as to be poised for rapid action upon insulin stimulus. Diffusional constraints are expected to be greatly exacerbated in an in vitro assay in which the cellular components are diluted by several orders of magnitude relative to the native intracellular milieu. Thus, the PI 3-kinase-dependent Akt activation in our in vitro system is likely to reflect the preservation of signaling compartmentalization that takes place in vivo at the interface between the membrane lipid bilayer and the aqueous phase.

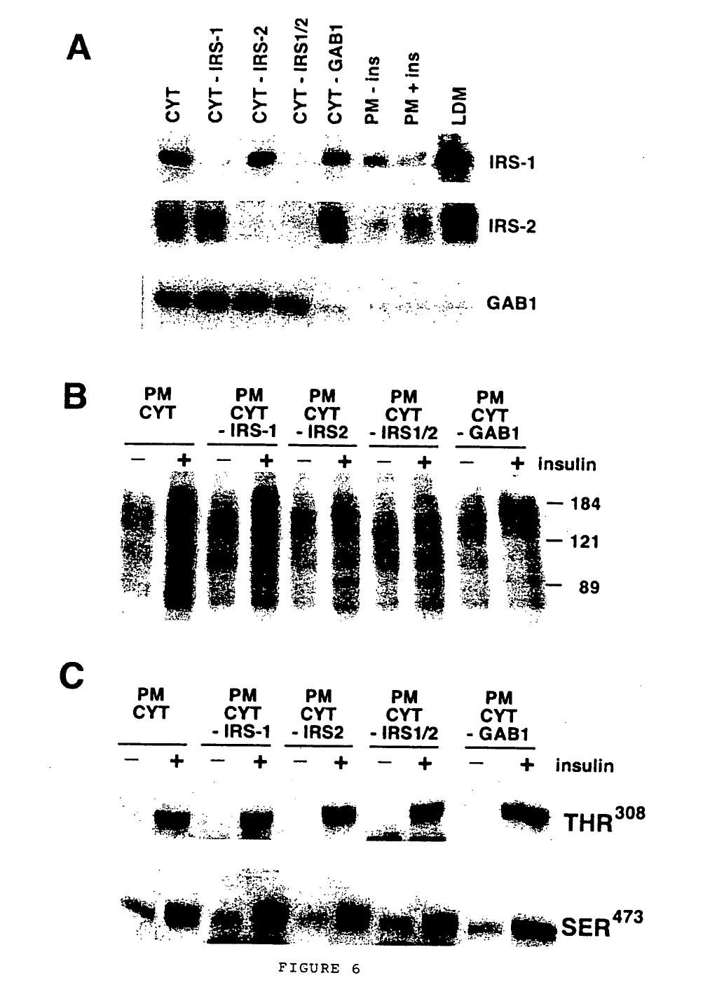

Soluble adaptor proteins could be uncoupled from downstream signaling using another approach. As shown in FIG. 6A, immunodepletion of CYT resulted in the successful removal of Gab1, IRS-1, IRS-2, or both IRS-1 and IRS-2 in combination. In vitro reactions were performed using PM(±)ins mixed with the various immunodepleted CYT. In the control reaction containing PM(+)ins and CYT mock-immunodepleted with an irrelevant antibody, the normal pattern of phosphotyrosine bands was observed by immunoblot (FIG. 6B). Closer inspection of the broad signal centered at ˜160 kDa revealed two bands in close apposition. Removal of IRS-1 from CYT resulted in the absence of the lower phosphotyrosine band, whereas removal of IRS-2 resulted in the absence of the upper phosphotyrosine band (FIG. 6B). This result is consistent with the slower reported electrophoretic mobility of IRS-2 relative to IRS-1. As expected, removal of both IRS-1 and IRS-2 from CYT (CYT-IRS1/2) resulted in the absence of the broad ˜160 kDa insulin-stimulated phosphotyrosine band (FIG. 6B). Removal of Gab1 from CYT did not noticeably alter the pattern of insulin-stimulated phosphotyrosine bands (FIG. 6B). Downstream signaling to Akt was then assessed by immunoblot using phospho-Akt antibodies. The removal of soluble adaptor proteins had no effect on insulin-stimulated Akt phosphorylation—both Thr308 and Ser473 were phosphorylated normally in all of the immunodepleted reactions (FIG. 6C). This experiment provides results complementary to that depicted in FIG. 5 and further reinforces the notion that IRS proteins in soluble form are not the conduits for productive signaling to PI 3-kinase in our system.

There are several possible explanations for these findings. IRS proteins may be entirely dispensable for signaling to Akt. Other adaptor proteins may be responsible for recruiting the PI 3-kinase activity necessary for Akt signaling. Alternatively, Akt signaling may be stimulated by a subpopulation of PI 3-kinase directly recruited to the activated insulin receptor (in a manner similar to that of other growth factor receptors). Finally, Akt may be activated by IRS proteins and PI 3-kinase already associated with the PM prior to insulin stimulation. In this regard, it is notable that readily detectable amounts of IRS-1, IRS-2, and p85 are reproducibly present in the PM derived from our fractionation protocol as demonstrated in FIG. 1B and FIG. 6A. This subpopulation of signaling molecules constitutively associated with the PM may be primed for immediate action following insulin stimulation. Other lines of investigation support the concept of compartmentalization in insulin signaling. For example, the expression of a membrane-targeted IRS-1 construct appears to inhibit cell proliferation but enhances signaling through Akt, despite less extensive insulin-stimulated tyrosine phosphorylation as well as dramatically decreased PI 3-kinase binding relative to wildtype IRS-1 (Kriauciunas, 2000). The exact site of action for the IRS proteins is uncertain. They appear to partition in a regulated manner between the cytosol and intracellular membranes (Inoue, 1998; Heller-Harrison, 1995). Their association with cytoskeletal elements has also been reported (Clark; 1998). Thus, the spatial organization of the various insulin signaling components in vivo still remains largely uncharacterized, but some of its features with regard to PI 3-kinase signaling appear to be intact in our in vitro system as evidenced by the efficient activation of Akt under our basic protocol conditions.

### Example 2

### In Vitro Phosphorylation of Ser473 of Protein Kinase B (Akt)

The preceding data demonstrated that early insulin signaling events dependent on PI-3 kinase, up to and including Akt and GSK-3 phosphorylation, appeared to be faithfully reconstituted with reasonable efficiency in our in vitro system. We utilized our system to investigate the molecular regulation of Akt, taking advantage of experimental manipulations made possible by unhindered access to all reaction components. One outstanding issue with regard to Akt regulation concerns the nature of the kinase activity, tentatively termed PDK2, responsible for phosphorylating Akt on Ser473 in the hydrophobic carboxy-terminal domain. At least three models for Ser473 phosphorylation have been proposed. Alessi and co-workers demonstrated that PDK1 could be converted in vitro, through interaction with a hydrophobic peptide (called PDK1-interacting peptide or PIF), into a form capable of phosphorylating Akt on both Thr308 and Ser473 (Balendran, 1999). Whether this unprecedented mode of regulation occurs in vivo remains unclear. Toker and Newton provided data supporting an Akt autophosphorylation mechanism involving the Ser473 site (Toker, 2000b), similar to that of certain conventional protein kinase C isoforms (Behn-Krappa, 1999). They suggested that Akt might be partially activated by phosphorylation of Thr308 due to upstream PDK1, thereby allowing Akt to act upon itself by transferring a phosphate group onto Ser473 (Toker, 2000b). Finally, it is possible for PDK2 to be a distinct kinase yet to be characterized. In cells lacking PDK1, growth factor-stimulated phosphorylation of Akt on Thr308 did not occur but phosphorylation of Ser473 still remained intact, suggesting th existence of a PDK2 kinase distinct from PDK1 (Williams, 2000).

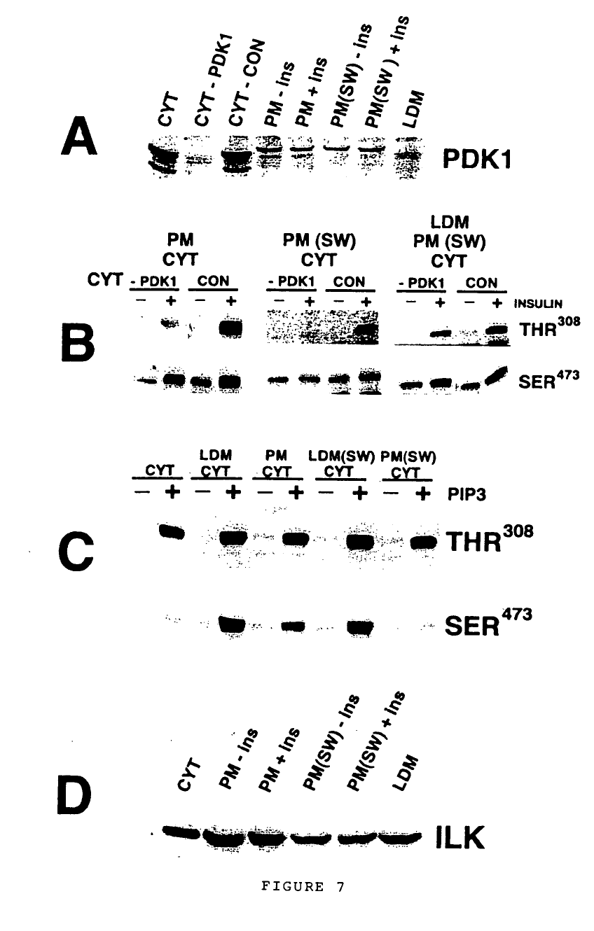

In order to clarify the role of PDK1 in the phosphorylation of Akt on Ser473, we performed our in vitro reaction using a CYT fraction from which PDK1 had been immunodepleted. Among the reaction components used, PDK1 was found predominantly in the CYT fraction (FIG. 7A), consistent with localization observed by others (Currie, 1999; Vanhaesebroeck, 2000). The faint band with a slightly retarded mobility observed in the PM fraction might be either a cross-reacting protein or a post-translationally modified form of PDK1. The LDM was essentially devoid of PDK1. Immunodepletion of CYT with an anti-PDK1 antibody successfully removed PDK1 (CYT-PDK1); in contrast, mock immunodepletion of CYT with an irrelevant antibody had no effect on PDK1 content (CYT-CON).

In an in vitro reaction combining immunodepleted CYT with PM, the lack of PDK1 resulted in greatly diminished insulin-stimulated phosphorylation of Akt on Thr308, as expected; however, insulin-stimulated Ser473 phosphorylation occurred normally (FIG. 7B, left panels). There is some evidence to suggest that the phosphorylation of Akt in vivo takes place following its recruitment to cellular membranes (Vanhaesebroeck, 2000). In order to address the possibility that PDK2 might be membran-associated, a reaction was performed by combining CYT with PM that had been washed with 1 M NaCl (PM(SW)). Salt extraction of PM abrogated insulin-stimulated Ser473 phosphorylation; however, insulin-stimulated Thr308 still occurred (FIG. 7B, middle panels). The inclusion of LDM to the reaction rescued insulin-stimulated Ser473 phosphorylation from being inhibited by salt extraction of PM (FIG. 7B, right panels).

Collectively, the data suggested that PDK2 appeared to be a kinase distinguishable from PDK1. This was strongly supported by the fact that independent manipulations (i.e. immunodepletion of PDK1 and salt extraction of PM) segregated these two kinase activities in complementary fashion (i.e. inhibiting PDK1 whereas leaving PDK2 intact, and vice versa). In contrast to the predominantly cytosolic localization of PDK1, PDK2 appeared to be associated with PM and LDM and largely absent from the cytosol.

The localization of PDK2 was also independently confirmed by another approach. We investigated whether the addition of exogenous PIP3 to our in vitro reaction could bypass the requirement for PI-3 kinase altogether, thus allowing the phosphorylation of Akt to occur in a non-insulin-dependent manner (FIG. 7C). Addition of PIP3 to CYT alone resulted in the efficient phosphorylation of Thr308, consistent with the localization of PDK1 predominantly in the cytosol; however, the phosphorylation of Ser473 occurred only marginally, consistent with PDK2 being largely absent from the cytosol. When PIP3 was added to a reaction containing CYT and LDM, the Thr308 signal was not further enhanced relative to that produced by a reaction containing CYT alone; in contrast, PIP3 addition to CYT and LDM produced a robust Ser473 signal, confirming the presence of PDK2 activity associated with the LDM. Heating the LDM at 65° C. for 10 min completely inhibited the PIP3-stimulated Ser473 activity suggesting that a catalytic protein was responsible for the PDK2 activity in the LDM rather than an ancillary thermostable cofactor (data not shown). Similar effects were observed when PIP3 was added to a reaction containing CYT and PM—i.e. the presence of PM significantly enhanced the phosphorylation of Ser473 but not Thr308. Salt extraction of the LDM did not affect Thr308 phosphorylation and only marginally reduced Ser473 phosphorylation. Approximately 47% of the total protein in the LDM could be extracted with 1 M NaCl. In contrast, salt extraction of the PM almost completely suppressed PIP3-induced Ser473 phosphorylation but had no effect on Thr308 phosphorylation. In this case, approximately 34% of the PM protein were removed with the salt wash. The data again supported the idea that PDK2, in contrast to PDK1, was mainly situated in membranes and absent in soluble form.

In addition to PIP3, we tested other phosphatidylinositol lipids for their ability to stimulate Akt phosphorylation. Only PIP3 and PI (3,4)P2 (but not PI (4,5)P2, PI (3)P, or PI) could stimulate Thr308 and Ser473 phosphorylation (data not shown). PIP3 and PI (3,4)P2 behaved identically in our system with respect to Akt phosphorylation.

Integrin-linked kinase (ILK) has been recently identified as a candidate for PDK2. The activity of ILK is apparently increased by insulin stimulation in a PI 3-kinase-dependent manner (Delcommenne, 1998). Using transfected cells, S. Dedhar and colleagues have provided evidence suggesting that ILK can phosphorylate Akt on Ser473 (Persad, 2001). However, the data regarding ILK are not conclusive. There is even uncertainty that ILK is a functional kinase-several critical residues normally found in the catalytic domain of protein kinases are not conserved in ILK (Lynch, 1999). These authors have concluded that ILK may regulate phosphorylation of Ser473 through an indirect mechanism (Lynch, 1999). To address the role of ILK in our system, immunoblot analysis using an ILK antibody was performed on 50 μg of each of the subcellular fractions (FIG. 7D). ILK was found enriched in the PM fraction relative to the CYT and LDM. However, the amount of ILK in the cytosolic fraction was significant considering the fact that our in vitro reactions typically contained five times the amount of cytosolic proteins relative to PM proteins. This observation is inconsistent with our expected profile for PDK2, which should be absent from the cytosol. In addition, although the amount of ILK in the PM was reduced with salt extraction, there was still a significant amount remaining. This behavior did not correlate with the PDK2 activity that we observed. These data indicate that the presence of ILK was not sufficient for Ser473 phosphorylation but they do not rule out the possibility that ILK may be necessary cofactor.

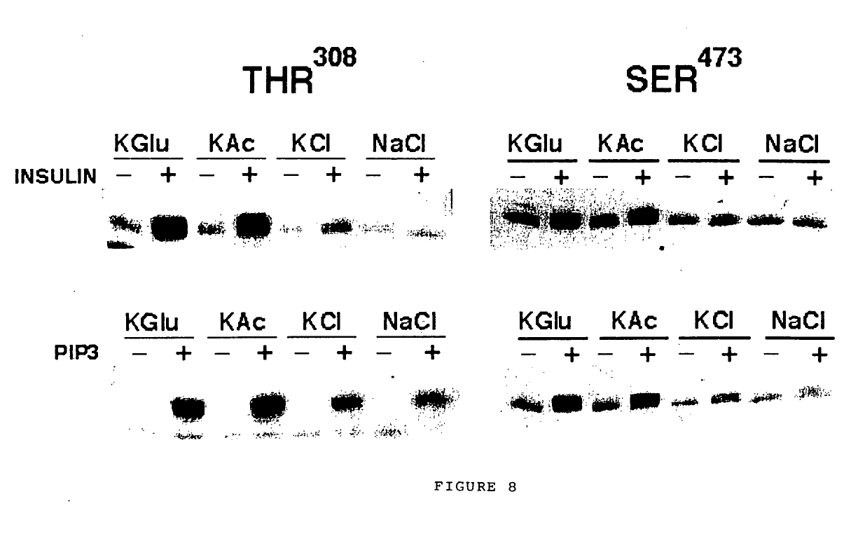

The definitive identification of PDK2 has remained elusive despite intense efforts by many investigators over the past several years (Brazil, 2001; Toker, 2000a; Vanhaesebroeck, 2000). The membrane localization of PDK2, which is herein described for the very first time, may have contributed to the technical difficulties experienced in attempts at purifying this activity. Initial efforts at reconstituting PDK2 activity from the salt extract of membranes were unsuccessful until it was discovered by the inventors that high concentrations of chloride (>100 mM) could completely inhibit PDK2 activity. This observation is illustrated in FIG. 8. In vitro reactions containing PM and CYT were carried out in the presence of 140 mM potassium glutamate/5 mM NaCl (KGlu), 140 mM potassium acetate/5 mM NaCl (KAc), 140 mM KCl/5 mM NaCl (KCl), or 145 mM NaCl. Insulin-stimulated phosphorylation of both Thr308 and Ser473 were almost completely suppressed in the presence of 145 mM chloride (FIG. 8, top panels). To address whether PDK1 and PDK2 activities were directly affected by chloride as opposed to an indirect effect involving an earlier signaling step, in vitro experiments were carried out using exogeneous PIP3 (FIG. 8, lower panels). The robust PIP3-induced Ser473 phosphorylation observed in the presence of potassium glutamate or potassium acetate was completely inhibited at high chloride concentrations. The PIP3-stimulated Thr308 phosphorylation was also severely inhibited with high salt but was apparent with longer film exposures. The effect of chloride is probably not observed in vivo since th intracellular concentration of chloride is low (approximately 4 mM). Nevertheless, buffers containing chloride in excess of 100 mM are routinely used in kinase assays and protein purifications, which may in part explain some of the past difficulty in identifying PDK2.

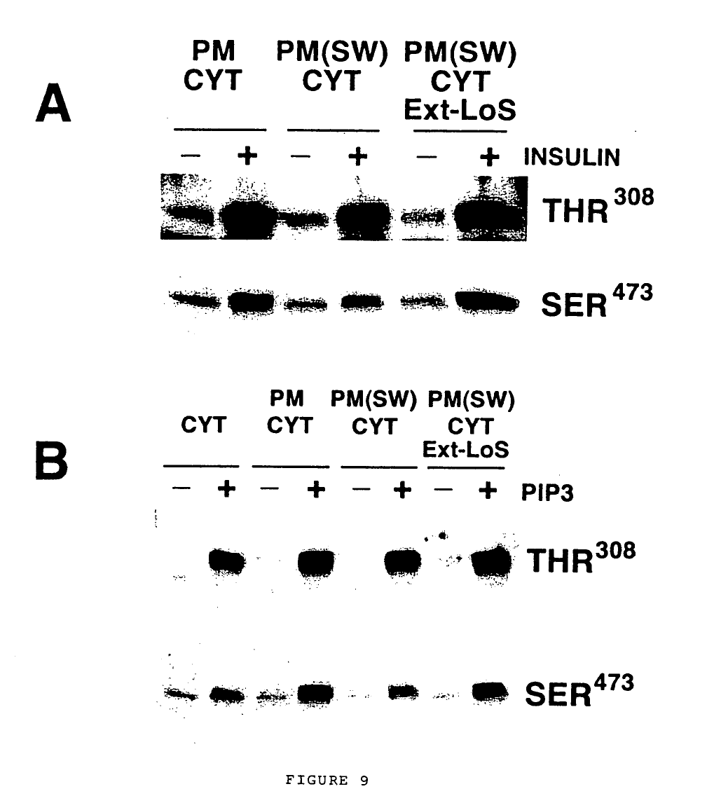

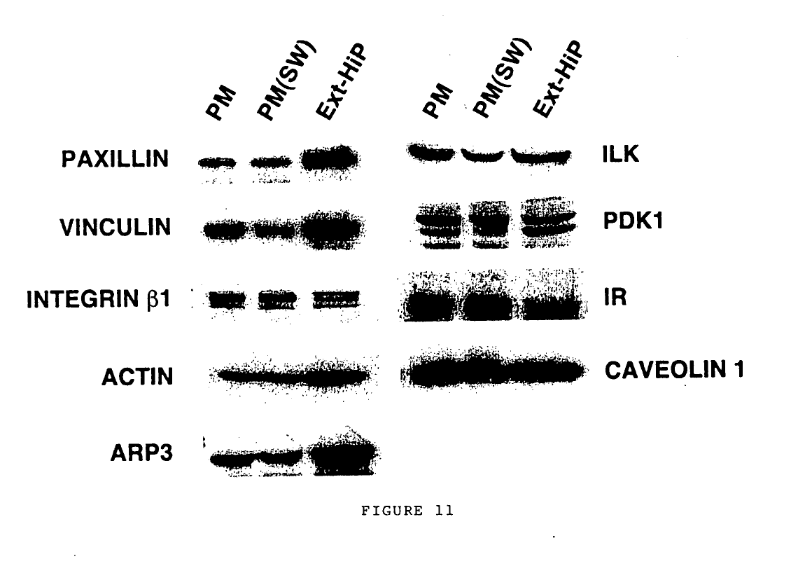

In order to address the possibility that PDK2 activity was irreversibly inhibited by 1 M NaCl as opposed to extracting the PDK2 activity from the PM, it was necessary to rescue the lost PDK2 activity by adding back the extracted proteins to the salt-washed PM. PM were salt-washed for 30 min in 1 M NaCl. The extracted proteins, that were recovered in the supematant after centrifugation at 37,000×g for 20 min, were desalted using a Sephadex G-25 spin column as described in General Methodology. As shown in FIG. 9A, salt washing of the PM almost completely inhibited insulin-stimulated phosphorylation of Ser473, but had no effect on insulin-stimulated Thr308 phosphorylation of PKBa/Akt1. Adding the desalted protein extract (Ext-LoS) back to the reaction containing the salt-washed PM and the cytosol recovered the PDK2 activity but had no effect on the PDK1 activity. The rescue result was confirmed using PIP3 to stimulate Akt phosphorylation (FIG. 9B). Again, washing the PM with high salt almost completely suppressed PIP3-induced Ser473 phosphorylation of PKBα/Akt1 but had little effect on Thr308 phosphorylation. Adding the extracted proteins back to the salt-washed PM and CYT recovered the stimulated-PDK2 activity with no effect on Thr308 phosphorylation. Thus, the data suggested that PDK2 could be dissociated from the bulk plasma membrane fraction by salt extraction in functional form. As shown in FIG. 1, PDK1 is mainly localized in CYT following our fractionation protocol. Its indisputably soluble nature is supported by the fact that it resists pelleting HiP by immunoblot analysis (FIG. 11) revealed that Ext-HiP was greatly enriched in paxillin, vinculin, actin, and the actin-associated protein Arp3. In contrast, this fraction appeared to contain less integrin β1 receptor than the PM and PM(SW) fractions. Interestingly, ILK, which has also been shown to be present in focal adhesion (Dedhar, 1999), was not enriched in Ext-HiP. A comparison between PM and PM(SW) indicated that 1 M NaCl removed some ILK from the membrane, but we found that like a typical peripheral protein, the ILK extracted with high salt predominantly localized to the Ext-HiS and not to the Ext-HiP (data not shown). Similar to our earlier observations, the distribution of ILK did not correlate with the observed PDK2 activity. The localization of PDK1 was ambiguous since PDK1 in the PM fraction appeared as two bands. The lower band, which comigrates with cytosolic PDK1, was enriched in Ext-HiP. The upper band, which represents the more abundant PDK1 signal in the PM, was de-enriched in the Ext-HiP. However we do not know whether the upper signal is due to a modified form of PDK1 or just a cross-reacting protein. The insulin receptor remained associated with the PM(SW) and was essentially absent from Ext-HiP. Caveolae are cholesterol-rich invaginations abundant in the plasma membranes of 3T3-L1 adipocytes and are important in the insulin-stimulated cbl-CAP pathway (Watson, 2001). Caveolin, a major protein in caveolae, however, was not enriched in Ext-HiP. We conclude from these results that the PM(SW) most likely contained the majority of the actual membrane comprising of the phospholipid bilayer, integral membrane proteins, and caveolae while Ext-HiP, the fraction that contained the bulk of the PDK2 activity in the PM, was enriched in cytoskeletal elements particularly focal adhesions. Incubation in 1 M NaCl disrupted the association between the cytoskeleton and the plasma membrane thereby allowing them to be segregated by centrifugation. Colocalization of PDK2 in focal adhesions is consistent with the previous observation that the integrin receptor signal transduction pathway activates Akt (Khwaja, 1997).

| SEQ ID NO:1 | |
|---|---|
| Homo sapiens protein (PKBα/Akt1) | |
| MSDVAIVKEGWLHKRGEYIKTWRPRYFLLKNDGTFIGYKERPQDVDQREAPLNNFSVAQC | 60 |
| QLMKTERPRPNTFIIRCLQWTTVIERTFHVETPEEREEWTTAIQTVADGLKKQEEEEMDF | 120 |
| RSGSPSDNSGAEEMEVSLAKPKHRVTMNEFEYLKLLGKGTFGKVILVKEKATGRYYANKI | 180 |
| LKKEVIVAKDEVAHTLTENRVLQNSRHPFLTALKYSFQTHDRLCFVMEYANGGELFFHLS | 240 |
| RERVFSEDRARFYGAEIVSALDYLHSEKNVVYRDLKLENLMLDKDGHIKITDFGLCKEGI | 300 |
| KDGATMKTFCGTPEYLAPEVLEDNDYGRAVDWWGLGVVMYEMMCGRLPFYNQDHEKLFEL | 360 |
| ILMEEIRFPRTLGPEAKSLLSGLLKKDPKQRLGGGSEDAKEIMQHRFFAGIVWQHVYEKK | 420 |
| LSPPFKPQVTSETDTRYFDEEFTAQMITITPPDQDDSMECVDSERRPHFPQFSYSASGTA | 480 |

1. An in vitro method of activating protein kinase B comprising
(a) obtaining from an insulin-responsive cell a membrane fraction and a cytoplasmic fraction, which comprises a protein kinase B.
(b) combining the membrane fraction, the cytoplasmic fraction and ATP in a buffer comprising less than 145 mM chloride, wherein
(c) the protein kinase B is activated by virtue of having a threonine residue and a serine residue phosphorylated, such that
(d) the activated protein kinaset B is capable of phosphorylating a GSK3.

2. The method of claim 1 wherein the insulin-responsive cell is treated with insulin.

3. The method of claim 2 wherein the membrane fraction is a plasma membrane fraction.

4. The method of claim 1 wherein the serine residue is at a position corresponding to amino acid 473 of SEQ ID NO:1 and the threonine residue is at a position corresponding to amino acid 308 of SEQ ID NO:1.

5. The method of claim 1 further comprising the step of combining PIP3 or PI(3,4)P2 with the membrane fraction, the cytoplasmic fraction and ATP in a buffer comprising less than 145 mM chloride.

6. The method of claim 5 further comprising the step of combining PIP3 with the membrane fraction, the cytoplasmic fraction and ATP in a buffer comprising less than 145 mM chloride.

7. The method of claim 1 wherein the insulin-responsive cell is a muscle cell, a liver cell, an adipocyte or an islet cell.

8. The method of claim 1 wherein the insulin-responsive cell is an adipocyte.

9. An in vitro method of activating protein kinase B comprising
(a) obtaining from an insulin-responsive cell a plasma membrane fraction and a cytoplasmic fraction, which comprises a protein kinase B,
(b) treating said plasma membrane fraction with a solution comprising at least 145 mM chloride, thereby obtaining a salt-extracted plasma membrane fraction and an aqueous fraction,
(c) desalting the aqueous fraction thereby producing a desalted aqueous fraction comprising less than 145 mM chloride,
(d) combining the salt-extracted plasma membrane fraction, the cytoplasmic fraction, the desalted aqueous fraction, ATP, and a phosphatidylinositol phosphate molecule in a buffer comprising less than 145 mM chloride, wherein
(e) the protein kinase B is activated by virtue of having a threonine residue and a serine residue phosphorylated, such that
(d) the activated protein kinase B is capable of phosphorylating a GSK3.

10. The method of claim 9 wherein the serine residue is at a position corresponding to amino acid 473 of SEQ ID NO:1 and the threonine residue is at a position corresponding to amino acid 308 of SEQ ID NO:1.

11. The method of claim 9 wherein the insulin-responsive cell is a muscle cell, a liver cell, an adipocyte or an islet cell.

12. The method of claim 9 wherein the insulin-responsive cell is an adipocyte.

13. The method of claim 9 wherein the insulin-responsive cell is treated with insulin.

14. The method of claim 9 wherein the phosphatidylinositol phosphate molecule is a PIP3 or PI(3,4)P2.

15. The method of claim 9 wherein the phosphatidylinositol phosphate molecule is a PIP3.

16. An in vitro method of phosphorylating a serine of protein kinase B comprising
(a) obtaining from an insulin-responsive cell a membrane fraction and a cytoplasmic fraction, which comprises a protein kinase B,
(b) combining the membrane fraction, the cytoplasmic fraction and ATP in a buffer comprising less than 145 mM chloride, wherein
(c) the protein kinase B is phosphorylated at a serine residue.

17. The method of claim 16 wherein the serine residue is at a position corresponding to amino acid 473 of SEQ ID NO:1.

18. An in vitro method of identifying an agent that modulates insulin activity comprising
(a) obtaining from an insulin-responsive cell (i) a membrane fraction, which comprises a phosphatidylinositol(3,4,5)P₃-dependent protein kinase-2 (“PDK2”) activity and an insulin receptor, and (ii) a cytoplasmic fraction, which comprises a protein kinase B and a phosphatidylinositol(3,4,5)P₃-dependent protein kinase-1 (“PDK1”) activity,
(b) combining the membrane fraction, the cytoplasmic fraction and ATP with the agent in a buffer comprising less than 145 mM chloride, and
(c) assessing the phosphorylation status of the protein kinase B.

19. The method of claim 18 wherein the insulin-responsive cell is treated with insulin.

20. The method of claim 18 wherein the membrane fraction is a plasma membrane fraction.

21. The method of claim 18 wherein the serine residue is at a position corresponding to amino acid 473 of SEQ ID NO:1 and the threonine residue is at a position corresponding to amino acid 308 of SEQ ID NO:1.

22. The method of claim 1 further comprising the step of combining PIP3 or PI(3,4)P2 with the membrane fraction, the cytoplasmic fraction and ATP in a buffer comprising less than 145 mM chloride.

23. The method of claim 24 further comprising the step of combining PIP3 with the membrane fraction, the cytoplasmic fraction and ATP in a buffer comprising less than 145 mM chloride.

24. The method of claim 18 wherein the insulin-responsive cell is a muscle cell, a liver cell, an adipocyte or an islet cell.

25. The method of claim 18 wherein the insulin-responsive cell is an adipocyte.

26. The method of claim 18 wherein the phopshorylation status of protein kinase B is assessed by immunoblot analysis using phospho-Akt antibodies.

27. A composition comprising a prepared membrane fraction obtained from a cell, wherein said prepared membrane fraction comprises an enzyme having PDK2 activity, wherein said PDK2 activity includes the phosphorylation of a serine residue of protein kinase B.

28. The composition of claim 27 wherein the cell is an insulin-responsive cell selected from the group consisting of islet cell, muscle cell, liver cell and adipocyte.

29. The composition of claim 27 wherein the cell is an adipocyte.
